(19) United States
(12) Patent Application Publication
(10) Pub. No.: US 2016/0367190 A1
(43) Pub. Date: Dec. 22, 2016

(54) METHOD AND SYSTEM OF CONTINUOUS MONITORING OF BODY SOUNDS VIA WEARABLE WIRELESS BODY SOUND MONITOR

(71) Applicant: MONDEVICES INC., North Bergen, NJ (US)

(72) Inventor: Arturas Henrikas Vaitaitis, North Bergen, NJ (US)

(21) Appl. No.: 14/740,883

(22) Filed: Jun. 16, 2015

(51) Int. Cl. A61B 5/00; A61B 7/02; A61B 7/04

(52) U.S. Cl. CPC A61B 5/6804; A61B 7/04; A61B 7/026; A61B 5/7282; A61B 5/746; A61B 5/6898; A61B 5/7203; A61B 5/7246; A61B 5/0004; A61B 5/0022; A61B 2560/0475; A61B 2560/0247; A61B 2560/0443; A61B 2562/16

(57) ABSTRACT

A method and system of continuous measuring, monitoring and analyzing sounds from person's body by a wearable wireless sound sensor worn or attached to clothing in close proximity to skin. The method and system include a wearable sensor including a universal attachment of the sound sensor to a user's clothing close or next to their skin in order to perform auscultation and analyze sound signals of the person over any durations of time. A mobile device in communication with the body sound sensor can analyze the collected measured sounds in order to create derived statistics based on received sound during any spans of time including large time intervals.

### BACKGROUND

Field of the Invention

The invention relates to medical monitoring of body sounds (i.e., auscultation). More particularly, it relates to a method and system for monitoring and measuring the sounds of a living body in real time using a wearable sensor.

Discussion of Related Art

The concept of listening to body sounds using a stethoscope is very well known. However, regular stethoscopes require both contact with the body and a human being listening to the same contemporaneously with the contact with the body. To do this manually is simply not cost effective and very intrusive to the patient or individual's everyday life. A regular stethoscope has very low volume and is significantly affected by ambient sound. A regular stethoscope does not allow storing any data. Therefore data cannot be correlated or analyzed. As such, the user of wireless monitors and the benefits of the same become readily apparent.

The main benefit of wireless technologies is ability to measure sounds at a great distances from a doctor over any durations of time. No longer is the presence of a doctor is required, a person could wear a wireless stethoscope during day and night in the comfort of home and upload the collected data to a database. The collected data then could be shared with an expert of medical profession or analyzed by a computer program.

An ability to collect data over significant durations of time requires substantial degree of convenience. Typical hospital conditions, where a presence of doctor is required or where a patient to be attached to wired sensors and lie on hospital bed limit duration of measurements due to costs and practical limitations. This invention focuses on method of attachment of a sound sensor or plurality of sound sensors that is convenient to patients and persons, namely attaching to tightly worn clothing, otherwise not different from T-shirts, bras or shirts. Alternatively a single sound sensor or multiple sound sensors could be attached to an adhesive tape that attaches directly onto a skin of a person. This degree of convenience would facilitate duration and quality of measurement of the body sounds that could be done without interfering with everyday activities of a person.

Wireless communication has been allowing increased patient mobility replacing physical devices cables for decades. Portable wearable monitors are becoming ubiquitous instruments in remote health-care. An introduction of low energy technologies, such as Bluetooth or similar wireless technology, alleviate some of the toughest constraints on power consumption facing portable medical devices, which limit their application.

With the ever increasing ubiquity of wireless sensors, an ease and universal nature of placement of the sensor as close as possible to the area of interest on a body for reliable readout becomes even more important.

The idea of monitoring body sounds on a regular basis, or constantly has very wide applications. However, a very good example of the area where it can make a significant impact is with persons that may have a difficulty communicating verbally health problems, such as kids and infants with asthma below the age of three or before they have learned to speak.

Children with asthma conditions under the age of 3 that have not learned how to speak would lack the ability to complain to parents. As a result this dangerous condition could go on without diagnosis for some time. With the present principles mentioned in this art, the parents will be able to monitor chest sounds of their children and either replay the records to the physicians or benchmark against normal sounds using an algorithm described in this art.

According to the CDC (http://www.cdc.gov/VitalSigns/asthma/) every one in 12 Americans had asthma in 2011, which affects 25 millions of Americans and the numbers increase every day. One in two people with asthma had an asthma attack in 2008. Among them, children and toddlers before the age of three that have not develop ability to communicate verbally and who develop asthma condition needs special care and monitoring. The children will not be able to verbally complain about the chest pain or conditions associated with difficulty in breathing. That task lies on caregivers and parents. If a child could complain, a caregiver or a parent can take him or her to a doctor. A doctor then could use a conventional medical tools, such as a regular stethoscope. In the absence of the ability to communicate verbally this becomes impossible. This is especially important to detect and start treatments of this dangerous condition early. This invention is trying to solve the problem of early diagnostic of asthma in children with wearable sensor technology, where the sound could be continuously collected and analyzed to programmatically detect dangerous conditions. The data could be recorded and transmitted to a practicing physician or a doctor.

There are many other segments of population that require constant monitoring that span beyond a short doctor visit. The ability to record and analyze a long stretches of time of a person's breathing sounds that span hours of day is currently impossible with conventional stethoscope that only lasts during a doctor visit and spans minutes.

Unlike a regular stethoscope, a solution described here will allow to store data and analyze it offline by a computer program. This would allow distinguishing any medical changes over long term periods. In case of a regular stethoscope, a patient has to rely on medical practitioner memory and interpretation of sounds.

Such condition may include, for example, lung sound monitoring, heart sound monitoring, monitoring of apnea and other lung or heart related conditions.

Wireless stethoscope inventions that address the problems with the use of a conventional Y-shaped doctor's stethoscope are known, where the sound is transmitted to the practitioners ear via Y-tubing. These problems include constraints to be in a proximity to a doctor and that the volume of sound traveling up the Y-tubing decreases with the distance. The numerous prior art addresses both of those problems by using modern electronic and wireless technology but leave another important problem. A regular stethoscope measurement is limited in duration by the timespan of a doctor visit.

This and other problem could be solved by a convenient wearable sensor that can be attached to clothing and continue to perform auscultation outside of the doctor office. Unlike the present principles, the prior art solutions are not wearable and therefore require a patient or individual to hold or temporarily affix an auscultation piece to their body.

Sound recorded by a wearable stethoscope may by optionally modified in order to enhance its quality and replicate the sounds of traditional stethoscopes for a more seamless transition for doctors to use a wearable stethoscope.

One of ways to enhance the quality is to apply noise reduction algorithms programmatically. The noise reduction will utilize sound recorded from plurality of sound sensors that differ from a sound sensor facing the body. The ambient sound will be subtracted from body sound providing a much clear signal.

### SUMMARY

According to an implementation, the method for monitoring body sounds includes providing a wearable sensor adapted to be in close proximity to or in contact with the a user's skin. The sensor monitors body sounds measured by the sensor through the user's skin and stores the measured body sound data in a database. The stored body sound data is processed to identify any irregularities. It is then determined if any identified irregularities meet or exceed a predetermined threshold, and an alert is triggered on a user's mobile device if any identified irregularity meets or exceeds the predetermined threshold.

According to another implementation, the system for monitoring body sounds of a user includes a wearable body sound sensor or plurality of sound sensors releasably attachable to the user's clothing so as to place the body sound sensor in close proximity or in direct contact with the user's skin. The sound sensor or multiple sound sensors are adapted to monitor body sounds and transmit signals relating to the monitored body sounds. A receiver is configured to receive the transmitted signals relating to the monitored body sounds and either retransmit such signals to another device, or process such signals to detect any irregularities that may be present in the same.

These and other aspects, features and advantages of the present principles will become apparent from the following detailed description of exemplary embodiments, which is to be read in connection with the accompanying drawings.

### BRIEF DESCRIPTION OF DRAWINGS

The invention is illustrated in the figures of the accompanying drawings, which are meant to be exemplary and not limiting, and in which like references are intended to refer to like or corresponding parts.

FIG. 1 is a schematic diagram of the system for monitoring body sounds using a wearable body sound sensor or plurality of sound sensors, according to an embodiment of the present principles;

FIG. 2 is a block diagram of portions of the mobile and sensors device sound monitoring system according to an embodiment of the present principles;

FIGS. 3 and 4 are schematic diagrams showing the attachment of the wireless body sound sensor to a material worn by the user, according to an embodiment of the present principles;

FIGS. 5 and 6 are schematic diagrams showing an alternative method of attaching the wireless body sounds sensor to a material to be worn by the user, according to an embodiment of the present principles;

FIGS. 7 and 8 are schematic diagrams showing another alternative method of attaching the wireless body sound sensor to a material to be attached directly to skin of the user, according to an embodiment of the present principles; and

FIG. 9 is a flow diagram of the method for monitoring body sounds using a wearable body sound sensor, according to an embodiment of the present principles.

### DETAILED DESCRIPTION

The present principles are directed to monitoring body sounds for medical purposes. The monitoring of the sounds of a living body is also known as auscultation.

The present description illustrates the present principles. It will thus be appreciated that those skilled in the art will be able to devise various arrangements that, although not explicitly described or shown herein, embody the present principles and are included within its spirit and scope.

All examples and conditional language recited herein are intended for pedagogical purposes to aid the reader in understanding the present principles and the concepts contributed by the inventor(s) to furthering the art, and are to be construed as being without limitation to such specifically recited examples and conditions.

Moreover, all statements herein reciting principles, aspects, and embodiments of the present principles, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the block diagrams presented herein represent conceptual views of illustrative circuitry embodying the present principles. Similarly, it will be appreciated that any flow charts, flow diagrams, state transition diagrams, pseudocode, and the like represent various processes which may be substantially represented in computer readable media and so executed by a computer or processor, whether or not such computer or processor is explicitly shown.

The functions of the various elements shown in the figures may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (“DSP”) hardware, read-only memory (“ROM”) for storing software, random access memory (“RAM”), and non-volatile storage.

According to an implementation of the present principles, a wearable sound sensor solves the problem with distance and sound volume, and also allows a continuous monitoring of sound over any durations of time, during or outside of doctor visits.

The present principles introduces a novel design for the universal attachment of a non-restrictive wearable sensor to a tight clothing or directly on the skin in a position best suited for measurement of living body sounds and vital health signals, such as but not limited to body movements, activity levels, heart rate, blood oxygen level and temperature.

The sensor unit is worn on a skin or on snugly fitted inner clothing, providing a skin contact. The method of attachment makes it convenient and unnoticeable for long durations of measurements. The measured sound is transformed into an electronic signal and is transmitted to an electronic reader, such as a personal smartphone or a wireless base. The signal can then be further retransmitted to a computer in a cloud, where the sound could be accessed or listened to at great distances from a source.

The present principles also relate to a method and a system of analysis of measured body sound to derive vital and health stats of a person who is wearing the units. The sound sensor, for example a microphone, is attached to the clothing or to an adhesive tape facing skin in order to get best quality measurements.

Measured signals are transmitted via wireless radio communication technology, such as, but not limited to, Bluetooth, radio frequency transmissions or similar technology to an electronic signal reader or plurality of signal readers. A wearable sensor may measure the following health parameters, including but not limited to: breathing; heart beats; pulse; lung noises, including asthma sounds; sounds of stomach and digestive track; sounds of a joint; and/or muscle movements.

Continuous and reliable measurements of the body sounds require specific placement of the sensor in the vicinity to the parts of the body that are being measured, in particular in the close proximity to skin. The present principles describe a method and a system to attach the sensor to persons clothing in the position best suited for the measurements.

Measurement and monitoring of sound for extended periods of time also require flexibility and convenience of attachment of an sound sensor

Measured data is transmitted to a plurality of electronic signal readers including but not limited to a computer, proprietary reader device, a tablet or a smart phone. A sensor allows measuring and detection of normal levels of vital signals, and in case signals are outside the defined norm, the sensor will issue an alarm notification to an electronic reader that will allow a person to take action. The sensor measurements may be transmitted further by a reader, acting as a pass through, to a remote computer database for storage, sharing and further analysis.

The present principles describe method of attachment of the sound sensors, i.e. microphones/stethoscopes, to the clothing of the person in the proximity to the body or to an adhesive material directly on skin.

In summary, the present principles defines a method of measuring the sounds of a living body by a wearable sensor in a way to successfully extract the best sound signal, the enclosure design of a wearable sensor unit that ensures an attachment to clothing near or at the body of a person facing the direction of skin, a method and a system to analyze the collected information.

Embodiments of the present principles disclosed herein have particular application to attachment of wearable sensors that measure sounds and health vital signs and use collected data to derive health status of a subject, such as but not limited to newborn babies, elderly subjects or subjects that require health care, and transmitting data to a mobile reader device, communication and interaction between a sensor unit and a mobile reader device. Yet such embodiments have application to interaction of several mobile, sensor and other devices on external environments of various kinds besides portable health monitoring, e.g., emergency, medical, sports and gaming, government and/or other kinds of systems, as long as one could measure the signal of the subject quantitatively via sensors attached to articles of clothing.

The following definitions are used to describe the details of the present principles and implementation of the same. Embodiments of the present principles utilize two parts of a wearable sensor: an external enclosure; and an internal sensor housing that is inserted into an enclosure. A wearable sensor worn by a subject communicates with a mobile reader device.
Universal attachment refers to ability to attach a wearable sensor:
   a) to inner article of clothing worn by the subject that provide a near access to skin; or
   b) directly on a skin, providing a method of attachment of external enclosure directly on subjects skin.
Internal sensor housing contains internal sensor parts, most notably a sound sensor, such as a microphone, fitted into the enclosure.
External sensor enclosure is a separate plastic part that ensures attachment of a sensor-unit to an inner article of clothing or directly onto skin. Clothing material is being sandwiched between an internal housing and an enclosure.
An internal sensor can also be attached an adhesive tape that, in turn, attaches directly to skin of a person with the sound sensor facing skin.
A sensor internal housing is inserted into a sensor enclosure with a clothing material in between. Thickness of a material ensures a secure firm attachment.
Sensor unit is a compact measuring device that monitors sounds and vital signs. It is compact enough to be located or worn around the body of a subject in a manner that is unobtrusive, does not restrict blood flow and allows reliable measurements of sounds and various status signals. Specific application requirements dictate that this device should be worn near body areas specific to acquisition of health signals, for example breathing monitoring. Embodiments of the present principles describe one sensor unit but those of skill in the art will appreciate that the system will work in similar manner with a plurality of sensor units.
Mobile device is a communication and computing device with a user interface and algorithms running on an operating system that is capable of detecting and capturing data from a sensor unit and retransmitting data further to be stored in a health database.
User-operator is a person who attaches a sensor-unit, responsible for the placement and adjustment of a sensor-unit on subjects articles of clothing. In certain cases, such as self attachment, a user-operator and a subject could be one and the same subject.

The goal of the present principles is to provide a secure and universal attachment of a wearable sensor on inner article of clothing that provides direct access to skin or directly on a skin in order to measure sounds and accompanying vital health signs of the subject, such as, but not limited to, heart rate, level of the oxygen in the blood and the temperature.

For a successful measurement of living body sound, a user-operator of a sensor-unit needs to place it in a certain position on subjects' body.

A universal attachment is achieved by placing clothing and/or adhesive material in between an internal housing enclosure and an external sensor enclosure and by inserting an internal housing into an external enclosure, until both enclosures trap clothing and/or adhesive material in between forming a secure attachment. A thickness of material will keep the attachment secure.

The present principles describe a universal attachment design of internal housing and external enclosure that allows flexible configuration of attachment, where internal housing could be either on top or at the bottom. The design of enclosure makes this possible because external housing has a large opening that allows direct contact of a microphone located on internal housing, even when internal housing is on top and external enclosure at the bottom. This is done for flexibility and convenience of attachment.

A mobile reader device will scan and detect a sensor unit in its operational vicinity and establish a communication session. If a sensor is present within the area of detection, a mobile device will establish a contact session with a sensor unit, will read health data measured by a sensor unit. In the absence of a sensor-unit, a mobile device will continue scanning for the presence of a sensor unit in its operational vicinity.

Measured signals can be optionally stored in a database outside the mobile reader device. In such case, a mobile reader will serve as a pass through and the device may transmit the data via a third communication protocol, not constrained by power consumption restrains, such as but not limited to WIFI or cellular signal. The stored data could be used for storage, sharing and more thorough analysis.

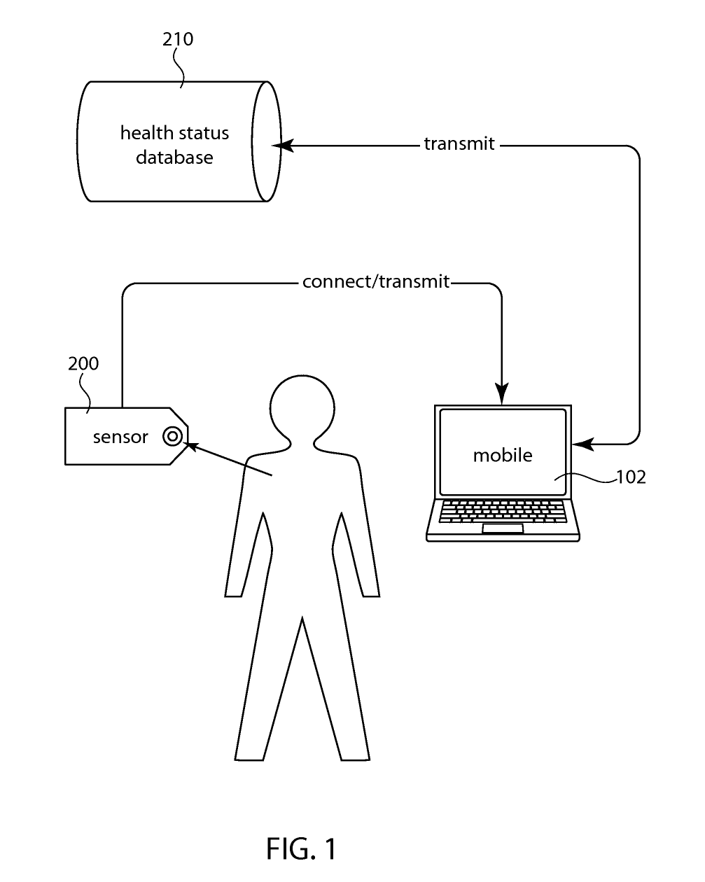

FIG. 1 illustrates a schematic description of sound sensor or a microphone operation, where a sound sensor 200 is attached to the clothing of a person in near proximity to the skin, or alternatively adhered directly to the skin using an adhesive material. Sensor 200 is configured to transmit a measured signal or signals to a personal mobile device or a wireless hub 102. Wireless hub 102, in turn, re-transmits the measured data to a database server connect to a local or remote network (e.g., in the cloud), where the data is stored in a health status database 210.

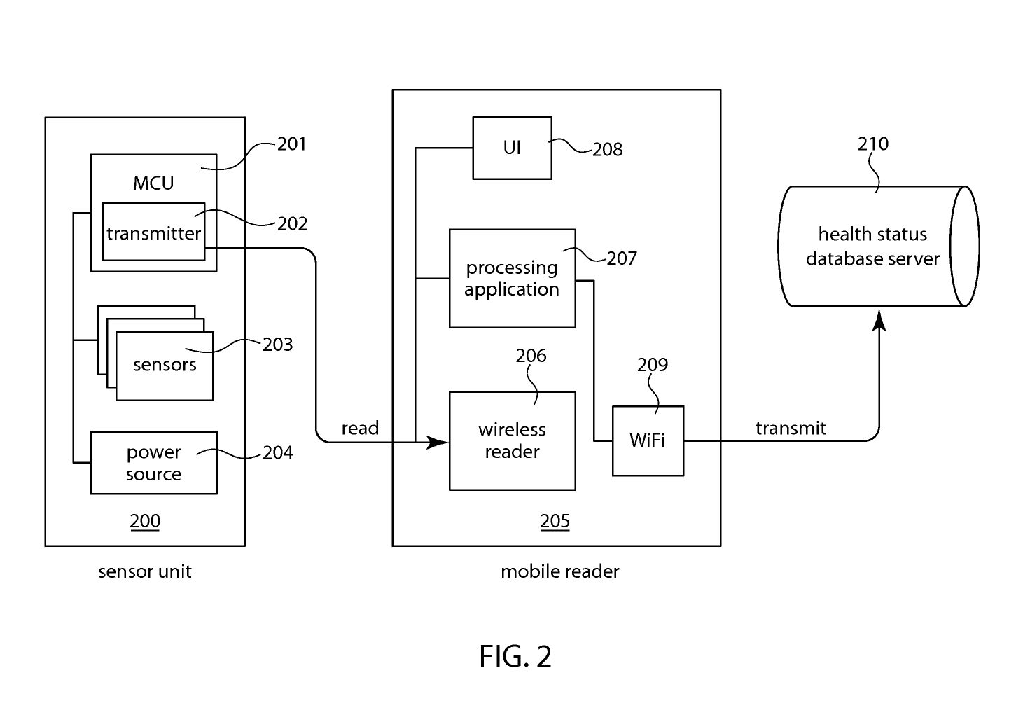

FIG. 2 is a schematic block diagram of portions of mobile and sensor devices sound monitoring system according to an implementation of the present principles. As illustrated, the sensor unit 200 consists of a micro-controller 201, a low energy wireless transmitter 102, at least one sound sensor or array of sensors 203 and a power source 204 that powers sensor unit components.

A mobile reader device 205 consists of several components specific for a mobile device, but components that are essential to the present principles are wireless low energy reader 206, a processing application 207 running within a processor (not shown for simplification purposes) and to be displayed on a user interface 208 as a front end to show sound signal and alerts. As represented in FIG. 2 the mobile reader device 205 detects and reads sensor unit 200, by receiving wireless low energy signals transmitted from the same. Mobile device 205 may re-transmit the signal further via WiFi interface 209 to a third-party sound database server located off-site 210.

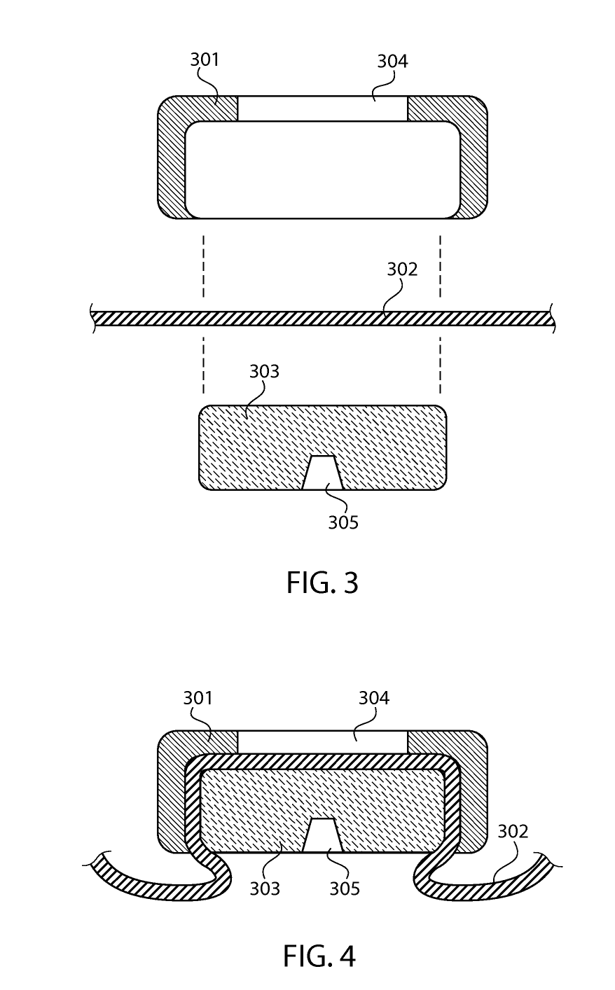

FIG. 3 depicts the configuration for attachment of a wearable sound sensor 200 to the article of inner clothing next to the subject body, in accordance with one implementation. As mentioned above, the sound sensor can also be attached to an adhesive material directly on skin to eliminate the need for

In accordance with this implementation, the sensor unit 200 includes an outer or external closure 301 and an inner or internal housing 303 containing the sensor 305. In operation, a user places some of their clothing material 302 in between the external enclosure 301 and an internal housing 303. Then the user presses the internal housing 303 into the external enclosure 301 until they will securely fasten, confining clothing there between (See FIG. 4). The internal housing 303 with sensor 305 will now be facing in the direction of the user's body, such that when the article of clothing is donned, the sensor 305 is in contact with the user's skin. As will be appreciated, due to the friction fit nature of the disclosed implementation, the thickness of the clothing 302 can have an effect on the strength of the attachment between the outer housing 301 and the inner housing 303. Thus, outer housing 301 can be configured to be more or less flexible depending on the anticipated thickness of clothing to which it would be connected.

FIG. 4 shows an example of the final stage of the operation of attachment. Here a wearable sound sensor 305 is attached to the article of clothing with the material 302 trapped (or friction fitted) between inner housing 303 and external enclosure 301. The direction of a microphone/sensor 305 will be facing the body/user's skin.

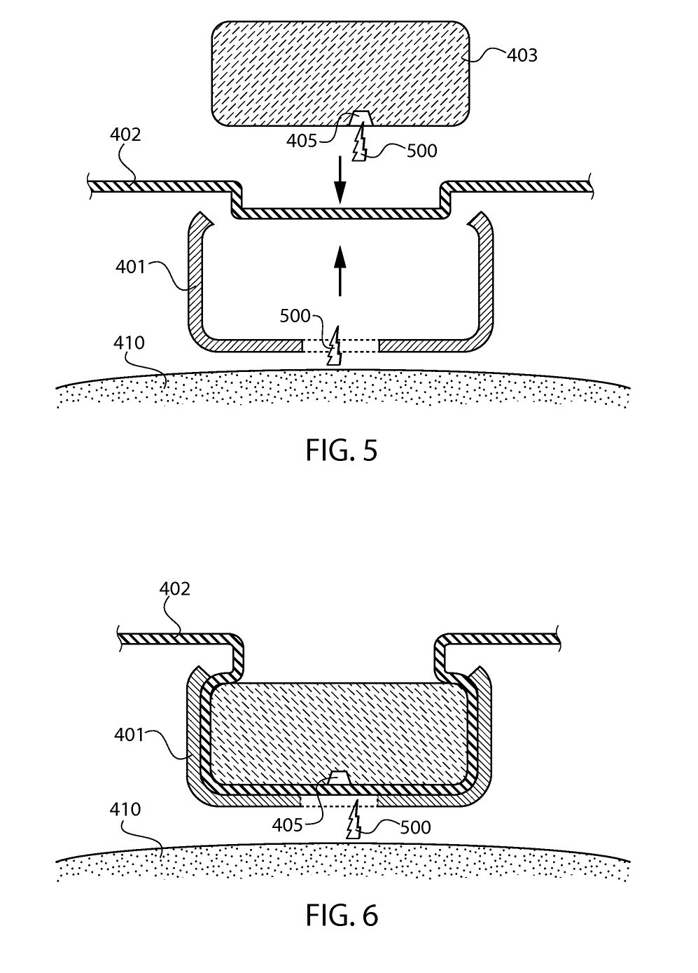

FIGS. 5 and 6 show an alternative configuration for attachment of a wearable sound sensor to the article of clothing in a near proximity to skin. The design of enclosure 401 allows this configuration of attachment, where the external enclosure 401 is at the bottom and the internal housing 403 of the sound sensor assembly with sensor 405 is on top. Note that the microphone/sensor 405 on internal housing is still facing the body. In this implementation, external housing/enclosure 401 has a large opening 404 that allows direct contact of the microphone/sensor 405 located in/on the internal housing 403, even when internal housing is on top and external enclosure at the bottom. This implementation makes attachment to a user's clothing 402 a very convenient process. FIG. 6 shows the assembled configuration with the clothing 402 positioned between the outer enclosure 401 and the inner housing 403 such that the microphone/sensor 405 can measure sounds 500 from the user's skin 410 via the very small gap formed by the opening 404 in the outer enclosure 401. In this configuration, the thickness of the user's clothing 402 may operate to dampen or attenuate body sounds as picked up from the microphone/sensor 405.

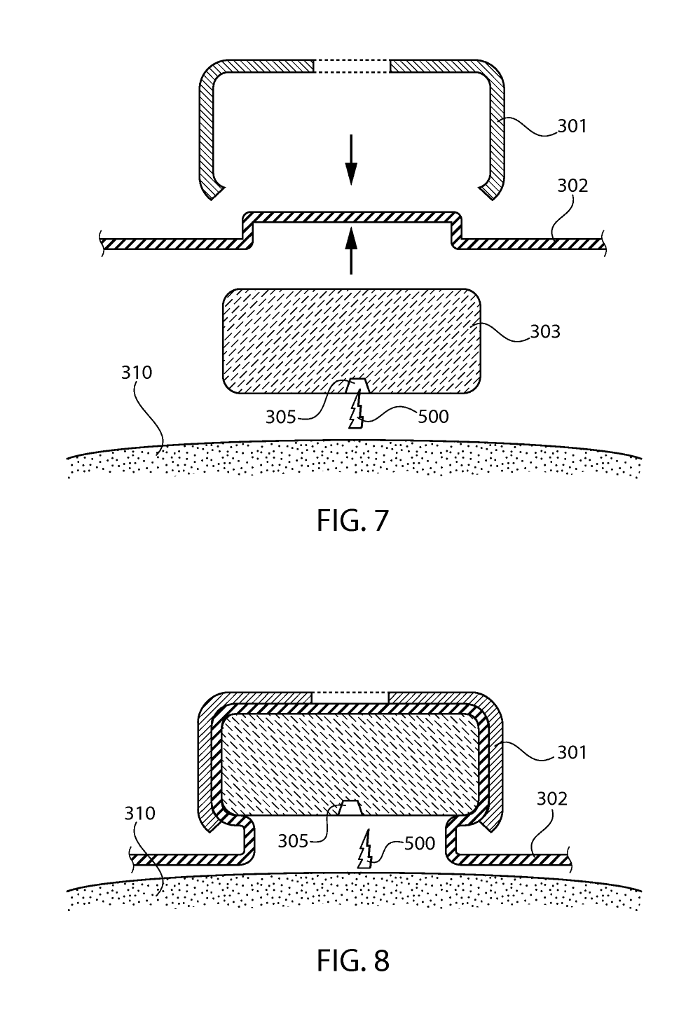

FIGS. 7 and 8 show an exemplary implementation of the sensor for measuring body sounds as shown in FIGS. 3 and 4. As shown, when the material 302 is positioned between the outer enclosure 301 and the inner housing 303, the microphone/sensor 305 can be in direct contact with the user's skin 310, and thereby measure the sounds therefrom. This is a preferred implementation due to the substantially direct contact of the microphone/sensor and the user's skin. As shown in FIG. 8, the proximity of the microphone/sensor 305 to the user's skin 310 is determined by the thickness of the material 402, but it will be appreciated that in this embodiment, microphone/sensor 305 will be substantially in contact with the user's skin 310.

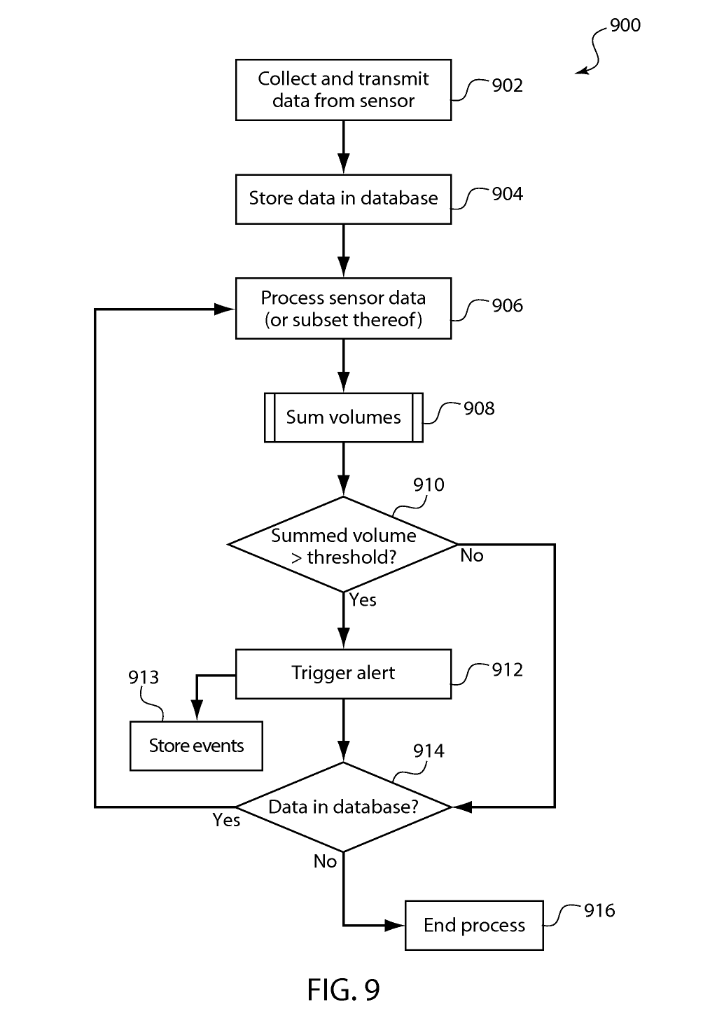

FIG. 9 shows a flow diagram of the method 900 for collecting and processing the sound data from the user. In one preferred implementation, the collected sound data 902 is stored in a database 904 and then processed by an algorithm (906-914) to detect and respond to interesting events, or for specific purposes. Those of skill in the art will appreciate that the simplest algorithm will divide data into datasets and process each dataset one at a time in a moving time window. At step 906, the sensor data is processed (or a subset thereof). This processing can include, for example, summing of the recorded volumes of body sounds (908), and then a determination as to whether the summed body sound volume meets or exceeds some predetermined threshold (910). If a threshold is reached or exceeded, the condition is marked/noted, and an audible and/or visual alert can be triggered (912) on the user's mobile device. An exemplary list of examples of events or irregularities that can be monitored includes sounds with the volume that exceed a specified threshold, sounds with the frequencies that fall outside of a specified frequency interval or a combination of both. The analysis of volume and frequency of measured sound could be as simple as a doctor listening to recorded sound or a computer program with machine learning technology. As mentioned, the triggering of an alert will also cause the same to be marked/recorded and stored (913) in the remote database (210) or the user's mobile device for later retrieval by a physician or other treating professional. After the alert is triggered, another determination is made (914) as to whether there is additional data is in the database, i.e., if there is sound data present in the database that has not been considered in the prior processing. If there is additional data in the database, the process restarts at step 906. If there is no additional data, the process can end (916).

A computer program may optionally modify amplitude and frequencies of recorded sound to programmatically or electronically enhance quality and replicate the sounds of traditional stethoscopes for a more seamless transition for doctors to use a wearable stethoscope. A computer program may optionally subtract the value of ambient sound recorded by a different sound sensor or plurality of sound sensors from the body sound recorded by sound sensor oriented toward the body, resulting in noise reduction. This process will calculate and assign weights to sound data based on individual sensor ID. Using a sensor ID one could correlate recorded data to an individual health vitals measured elsewhere.

The following is only one example of a computer program that demonstrates the analysis of sound measured by the sound sensor where signal levels are benchmarked against normal levels of sound. If the measured signal exceeds the configured thresholds, an event is set and triggered.

```
#define _USE_MATH_DEFINES
#include <stdio.h>
#include <stdlib.h>
#include <string.h>
#include <math.h>
#include “uthash.h”
#include “uniqhash.h”
#define HIGH_THRESHOLD 0.31
#define HIGH_THRESHOLD_BIT 3
double detect_motion1(double *bufx_ptr, double *bufy_ptr,
double *bufz_ptr, double *ttms_ptr, int wsize, double *distance,
int *event)
{
  int ii;
  double* vx = bufx_ptr;
  double* vy = bufy_ptr;
  double* vz = bufz_ptr;
  double* tt = ttms_ptr;
  int ilen = 0;
  double mean_disp = 0.0;
  for (ii = 0; ii < wsize; ii++)
  {
    if(tt[ii] <= 0 || tt[ii] > tt[wsize−1] − 3*NBUF1*NBUF2/FREQ1) {
      mean_disp += sqrt(vx[ii]*vx[ii] + vy[ii]*vy[ii] + vz[ii]*vz[ii]);
      ilen++;
    }
  }
  if(ilen <= 0) {
    return 0;
  }
  if(ilen > 0) mean_disp /= ilen;
  double disp = 0.0;
  for (ii = 0; ii < wsize; ii++)
  {
    if(tt[ii] <= 0 || tt[ii] > tt[wsize−1] − 3*NBUF1*NBUF2/FREQ1) {
      disp += fabs(sqrt(vx[ii]*vx[ii] + vy[ii]*vy[ii] + vz[ii]*vz[ii]) −
      mean_disp);
    }
  }
  *distance = disp;
  if(disp > HIGH_THRESHOLD) {
    (*event) |= (1 << HIGH_THRESHOLD_BIT);
  }
  return disp;
}
```

It is to be further understood that, because some of the constituent system components and methods depicted in the accompanying drawings are preferably implemented in software, the actual connections between the system components or the process function blocks may differ depending upon the manner in which the present principles are programmed. Given the teachings herein, one of ordinary skill in the pertinent art will be able to contemplate these and similar implementations or configurations of the present principles.

Although the illustrative embodiments have been described herein with reference to the accompanying drawings, it is to be understood that the present principles is not limited to those precise embodiments, and that various changes and modifications may be effected therein by one of ordinary skill in the pertinent art without departing from the scope or spirit of the present principles. All such changes and modifications are intended to be included within the scope of the present principles as set forth in the appended claims.

What is claimed is:

1. A method for monitoring body sounds comprising:
providing a wearable sensor or plurality of wearable sensors adapted to be in close proximity to or in contact with the a user's skin;
monitoring body sounds measured by the sensor or plurality of sensors through the user's skin;
storing the measured body sound data in a database;
processing the stored body sound data to identify any irregularities;
determining if any identified irregularities meet or exceed a predetermined threshold;
triggering an alert on a user's mobile device if any identified irregularity meets or exceeds the predetermined threshold.

2. The method according to claim 1, wherein said processing further comprises:
subtract the ambient sound recorded by a different sound sensor from the body sound, resulting in noise reduction
modify amplitude and frequencies of recorded sound signals to enhance the quality of the recorded sound
calculate weights to body sound signals based on recorded data parameters
summing all measured body sound levels with calculated weights; and
comparing the summed value to a predetermined threshold.

3. The method according to claim 1, wherein said providing a wearable sensor comprises providing a sensor having an outer enclosure and an inner sensor housing; the outer enclosure being removable from the inner sensor housing such that the user can position an article of their clothing between the outer enclosure and inner sensor housing and sandwich the clothing there between by forcing the outer enclosure around the inner sensor.

4. The method according to claim 1, further comprising:
determining whether the stored body sound data has changed since said processing; and
in the event the stored body sound data has changed, repeating said processing, determining and triggering based on the changed sound data.

5. The method according to claim 1, further comprising:
determining whether the stored body sound data has changed since said processing; and
in the event the stored body sound data has not changed, terminating the method.

6. A system for monitoring body sounds of a user comprising:
a wearable body sound sensor configured to attach to the user's clothing and place the body sound sensor in close proximity or in direct contact with the user's skin, the sound sensor being adapted to monitor body sounds and transmit signals relating to the monitored body sounds; and
a mobile device configured to receive the transmitted signals relating to the monitored body sounds and process such signals to detect any irregularities that may be present in the same, said mobile device being further configured to transmit received body sound signal data to a remote database configured to store the signals.

7. The system according to claim 6, wherein said mobile device comprises a processor that is configured to:
process the stored body sound data to identify any irregularities;
determine if any identified irregularities meet or exceed a predetermined threshold;
trigger an alert on the mobile device if any identified irregularity meets or exceeds the predetermined threshold.

8. The system according to claim 7, wherein said wearable sensor comprises:
an outer enclosure; and
an inner sensor housing;
the outer enclosure being removable from the inner sensor housing such that the user can position an article of their clothing between the outer enclosure and inner sensor housing and sandwich the clothing there between by forcing the outer enclosure around the inner sensor.

9. The system according to claim 7, wherein the processor is further configured to:
determine whether the stored body sound data has changed since said processing; and
in the event the stored body sound data has changed, repeat said processing, determining and triggering based on the changed sound data.

10. The system according to claim 7, wherein the processor is further configured to:
determine whether the stored body sound data has changed since said processing; and
in the event the stored body sound data has not changed, terminate the processing.

11. A system for monitoring body sounds of a user comprising:
a wearable body sound sensor releasably attachable to the user's clothing so as to place the body sound sensor in close proximity or in direct contact with the user's skin, the sound sensor being adapted to monitor body sounds and transmit signals relating to the monitored body sounds; and
a receiver configured to receive the transmitted signals relating to the monitored body sounds and either retransmit such signals to another device, or process such signals to detect any irregularities that may be present in the same.

12. The system according to claim 11, wherein said receiver comprises a processor configured to:
process the monitored body sound data to identify any irregularities;
determine if any identified irregularities meet or exceed a predetermined threshold;
trigger an alert on a device in communication with the user if any identified irregularity meets or exceeds the predetermined threshold.

13. The system according to claim 12, wherein said wearable sensor comprises:
an outer enclosure; and
an inner sensor housing;
the outer enclosure being removable from the inner sensor housing such that the user can position an article of their clothing between the outer enclosure and inner sensor housing and sandwich the clothing there between by forcing the outer enclosure around the inner sensor.

14. The system according to claim 12, wherein the processor is further configured to:
determine whether the stored body sound data has changed since said processing; and
in the event the stored body sound data has changed, repeat said processing, determining and triggering based on the changed sound data.

15. The system according to claim 12, wherein the processor is further configured to:
determine whether the stored body sound data has changed since said processing; and
in the event the stored body sound data has not changed, terminate the processing.
